(19) United States
(12) Patent Application Publication
(10) Pub. No.: US 2017/0079897 A1
(43) Pub. Date: Mar. 23, 2017

(54) COMPOSITION FOR HAIR APPLICATION AND METHODS OF HAIR CARE

(71) Applicant: Matthew Brissette MINUS, Houston, TX (US)

(72) Inventor: Matthew Brissette MINUS, Houston, TX (US)

(21) Appl. No.: 15/270,526

(22) Filed: Sep. 20, 2016

Related U.S. Application Data
(60) Provisional application No. 62/221,033, filed on Sep. 20, 2015.

(51) Int. Cl. A61K 8/44; A61Q 5/06; A61Q 5/12; A61K 8/92

(52) U.S. Cl. CPC A61K 8/44; A61K 8/922; A61K 8/925; A61Q 5/06; A61Q 5/12

(57) ABSTRACT

The present invention teaches all-natural formulations comprising amino acids for hair application. These formulations can be used for a wide range of hair treatments including hair detangling, hair curl loosening, hair straightening, for preventing hair breakage and stimulating hair growth. The methods used to generate the formulations are disclosed, along with the methods for treating hair with the formulations to attain one or more desired benefits.

### CROSS-REFERENCE TO RELATED APPLICATIONS

The present application claims benefit of U.S. Provisional Application Ser. No. 62/221,033 filed on Sep. 20, 2015; the entire contents of which are incorporated herein by reference.

### FIELD OF THE INVENTION

This product pertains to proper health and care of hair. More specifically it assists in the detangling, curl loosening, curl stretching, and straightening of hair.

### BACKGROUND OF THE INVENTION

Hair Conditioners

A standard hair conditioner is an emulsion of water and oil. The oil surrounds and embeds in the cuticle along with the water. The hair slowly absorbs the water leaving the oil coating on top. The oil layer creates a barrier between the atmosphere which locks in the moisture that was added to the hair. Many modern conditioners fail to find the correct balance of oil. If the oil is too heavy it creates a limp and stringy hair appearance. If the oil is too light the hair will appear light and soft, but the conditioner will not lock in moisture. For many years cosmetic chemists have struggled to find the balance between a conditioner that locks in moisture while still providing light, bouncy, and soft hair.

To remedy the balance between looks and hydration more modern conditioners have turned to non-volatile alcohols like glycerin, propylene glycol, sterol, and cetyl alcohol. These alcohols coat the cuticle giving it a shiny, lustrous and healthy appearance. They also act as preservatives to prevent mold formation in products. Unfortunately, they do not seal in moisture. In fact, these alcohols help to dehydrate the hair cortex from the inside out. Over time the hair cortex weakens and shrivels like a dried branch and the user begins to notice shedding/breakage. By embedding the hair cuticle with the proper balance of amino acids, the composition of the present invention locks in moisture without weighing the hair down.

Relaxers

Hair with a tight curl pattern often grows in a way that it becomes tangled and unmanageable. In order to remedy this problem, relaxers are often used, which contain hydroxides to detangle and straighten hair follicles. Unfortunately, the chemical harshness of hydroxides is incompatible with organic tissue causing chemical burns on the skin and irreversible damage to the hair follicle itself. Newer approaches for this involve keratin proteins and propylene glycol. In comparison to hydroxide-based relaxers, keratin relaxers are a step forward in the hair straightening industry, because they do not burn the skin. However, when heated, proplylene glycol turns into formaldehyde, a known carcinogen. In fact, these treatments are so toxic that they have been outlawed in California. Another drawback is that most keratin treatments only produce straight hair and do not allow access to the full array of curly hair patterns. Also, these protein-based straightening products generally use high temperature flat ironing to get more lasting results.

Very few attempts at natural amino acid relaxers have been have been successful. Most are heavy in salts and inappropriate for straightening African-American Hair.

A low-irritant, high performance composition for permanent waves has been previously described in Jin, J., (2011) US Patent Application No. US2011/0189119 A1, which operates at an inappropriately acidic pH (pH 5-7) for the straightening of tighter curl patterns, (3-4, a-c) and does not use lysine as a primary follicle stimulating amino acid. The composition disclosed in the cited reference also does not employ branched chain amino acids (BCAAs) for the proper hydration and moisture retention of the cuticle which in turn prevents hair breakage, and uses primarily cysteine instead of N-acetyl cysteine. Further, the composition disclosed in the above-mentioned reference describes the formation of a dry powder and does not specify the aqueous concentration. It has been recognized that the amount of water added is quintessential for the potential relaxing properties of the claimed invention.

An aqueous composition for the permanent shaping of keratinous fibers, especially human hair, has been described in Lissner et al., (2012) WO 2012/163594 A2. The composition described in this reference uses unnatural synthetic alkanolamines (not all-natural) for waving/straightening hair, and cysteine for the reduction of disulfide bonds.

### SUMMARY OF THE INVENTION

The composition of the claimed invention comprises natural amino acids (no proteins or hydroxides) including but not limited to branched chain amino acids and follicle stimulating amino acids at a pH that is most optimal to attain the desired beneficial effects on the hair without adverse effects on the scalp. In some embodiments, the pH can be buffered by alkali and/or alkaline earth phosphates, bicarbonate, and/or carbonates. The composition of the present invention is the first naturally occurring amino acid based relaxer that can not only straighten tight curl pattern, but it can also strengthen, stimulate hair growth, and prevent future breakage.

In some embodiments, the composition of the claimed invention is used as a conditioner. The claimed invention can embed amino acids into the hair that retain moisture and strengthen the hair cuticle. In contrast to heavy oil based conditioners, the claimed invention can keep the hair light, while in contrast to alcohol rich conditioners, it can keep the hair strong and properly hydrated.

In other embodiments, the composition of the claimed invention is used to hydrate, detangle, strengthen, loosen curls, straighten or a combination thereof hair with curl patterns 2a-c, 3a-c, 4a-c or hair with no defined curl pattern, without chemical burning or follicle damage. The mildness of the claimed formulations allows for attaining a wide array of desired curl patterns. Moreover since the claimed composition can be made using only all-natural ingredients that are healthy for hair, it is the first curl loosening agent that can be reapplied on a daily basis. In addition, the curl loosening and straightening application of this product does not require flat ironing.

In some embodiments, the composition further includes oil such as for example jojoba oil and/or emulsifying wax.

### BRIEF DESCRIPTION OF DRAWINGS

FIG. 1 shows representative examples of hair detangling effects and hair straightening effects of the claimed invention.

### DETAILED DESCRIPTION OF THE INVENTION

All references cited herein are incorporated by reference in their entirety. Unless otherwise defined, the scientific and technical terms used herein have the meaning as commonly understood by one of ordinary skill in the art. The present invention is not limited to the methods and formulations described below. It is understood that one of ordinary skill in the art can modify the disclosed methods, formulations and embodiments to attain alternative or variant embodiments with alternative or variant usages, all of which are covered by the present application and can be within the scope of the application. The following terms are defined below in the context of the present application.

“Hair” can refer to hair growing from the scalp or any part of the body. “Mammalian hair” refers to hair of a mammal including but not limited to human, horse, dog, cat, hamster, gerbil, guinea pig, and rabbits. Preferable embodiment comprises application of the composition to human hair of the scalp.

The terms “composition” and “formulation”, used interchangeably, refer to a product for application on hair. “Hair breakage” may refer to the breakage of a hair strand at any point along the length of the hair strand resulting in shorter hair strand length. “Hair shedding” may be used interchangeably with “hair loss” and can refer to the reduction of hair growth, reduction in the length of hair grown, falling out of hair or any combination thereof. “Injured” or “damaged” hair may be used interchangeably and can refer to hair strands that are reduced in strength and/or length due to prolonged and/or repeated exposure to chemicals, dyes, and/or heat. Injured hair can refer to hair that has been damaged by hair treatments such as perming and straightening and mechanical damage due to sub-optimal handling of hair. Injured hair includes hair strands that have split ends, mid-shaft splits, thinning edges and knots. “Tangled” hair typically refers to hair that cannot be brushed through, and may be a result of hair strands that have damaged cuticles. “Dry” hair refers to hair that does not have enough moisture and oil so as to exhibit normal sheen and texture. “Brittle hair” refers to hair prone to breakage and includes hair that is dry and frizzy or is otherwise in poor health. “Healthy hair” refers to hair that has sufficient strength to resist breakage at any point along the length of the hair strand, abundant moisture, elasticity, softness and shine, and does not exhibit hair loss, shedding, damage or injury.

Hair, particularly human hair growing from the scalp, has different textures—straight, wavy and curly among others. Curly hair may refer to hair that has an S-shape, while straight hair grows in a manner that is substantially without a bend or angle. Curl pattern has been classified into different categories. Curl pattern 4 refers to very tightly curled, coiled hair, which typically is more prone to breakage and damage from hair treatments. Hair of curl pattern 4 is further classified into curl pattern type 4a which has an “S” pattern and type 4b which has a “Z” pattern. Curl pattern type 4c is similar to type 4b with the curls being tightly kinked and with less definition. Curl pattern 3 includes hair that shows well-defined, springy curls. Curl pattern 3 can be further classified into curl pattern types 3a (definite loopy S pattern), 3b (smaller circumference than 3a curls) and 3c (curls resembling tight corkscrews). Hair of type 3c typically is coarser than hair of type 3b, which is in turn typically coarser than hair of type 3a. Curl pattern 2 includes hair of wavy texture, with type 2a being slightly “S” shaped wavy hair that is typically fine, type 2b being S-shaped having more defined curls than type 2a, and with type 2c being the coarsest and most curly of the type 2 hair patterns.

“Hair strengthening” refers to one or more processes by which hair attains reduced breakage, increased structural resilience, increased elasticity, increased rigidity and/or increased sheen. “Hair softening” refers to one or more processes by which hair attains increased hydration and/or increased shine. “Hair detangling” refers to one or more processes that help hair become less tangled, which can then be brushed more easily. “Hair curl loosening”, alternatively “hair curl stretching”, refers to one or more processes that reduces the curly texture of hair and/or increases the definition, moisture content and shine of curls. “Hair straightening” refers to one or more processes that alter the texture of hair such that the hair attains a more straight texture. “Hair growth” may refer to the growth of new hair strands and/or an increase in the length of hair strands. “Hair hydration” interchangeably used with “hair conditioning” may refer to an increase in, retention of, prevention of, loss of or a combination thereof the moisture content of the hair.

“Inorganic salt” refers to a chemical product of a reaction between an acid and a base, which does not contain carbon, such as for example, sodium chloride.

Effective amount of composition refers to an amount that results in one, few, most or all of the desired benefits from the use of the composition. The use of an effective amount of the composition may also result in varying degrees of a particular benefit.

“Preventing” refers to a delay in the onset of an event or condition, a reduction the severity of an event or condition or a combination thereof. The term may refer to the absence of the event/condition, however, it is not limited to the absence of the event/condition. “Reducing” may be used interchangeably with “alleviating” and refers to a decrease in the severity and/or presence of a condition over a period of time.

An amino acid has the structure shown in a) below and is defined by its side chain functional group (R). An example of the amino acid of the claimed invention is shown in b) below. An example of the claimed invention with carbonates/bicarbonates is shown in c) below.

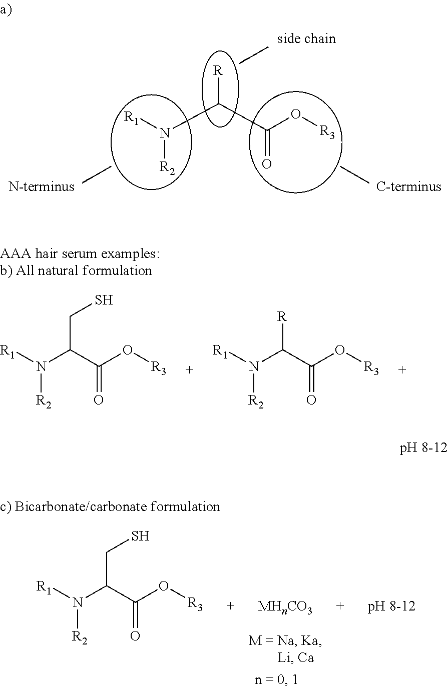

Water soluble derivatives can be made by adding the functional groups, such as for example, those listed in Table 1 to the N-terminus (R₁, R₂) and the C-terminus (R₃).

TABLE 1

| R₁ | R₂ | R₃ |
|---|---|---|
| H | H | H |
| 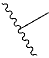 | 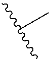 | 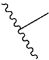 |
| 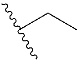 [SMILES: *CC] | 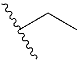 [SMILES: *CC] | 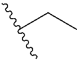 [SMILES: *CC] |
| 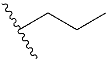 [SMILES: *CCC] | 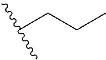 [SMILES: *CCC] | 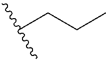 [SMILES: *CCC] |
| 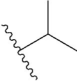 [SMILES: *C(C)C] | 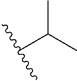 [SMILES: *C(C)C] | 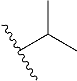 [SMILES: *C(C)C] |
| 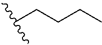 [SMILES: *CCCC] | 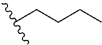 [SMILES: *CCCC] | 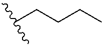 [SMILES: *CCCC] |
| 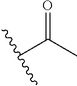 [SMILES: *C(C)=O] | 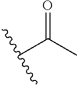 [SMILES: *C(C)=O] | |

Glycine (1) and some of its water-soluble derivatives (2-5) are shown below as an example of the potential agents that can be used in the claimed invention. They contain different combinations of R₁, R₂ and R₃ that are listed in Table 1.

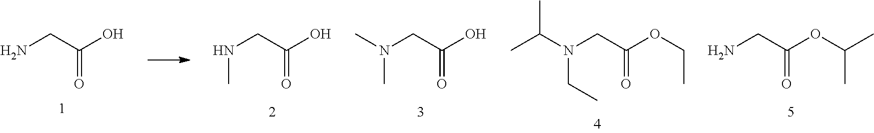

In a preferable embodiment, the composition of the present invention comprises an aqueous solution/suspension containing (1) one or more branched chain amino acids (leucine, isoleucine, valine) (5-15 parts) (2) one or more follicle stimulating amino acids L-Lysine (1-50 parts), N-acetyl cysteine (25-30 parts) and (3) deionized or distilled water (100 parts) to be used for hydration and enhanced growth of hair. In this context, parts are defined as equal units of mass.

The composition of the present invention is free, or substantially free, of added inorganic salts. Hair products comprising high salt cause diffusion of water molecules from the inner shaft of the hair into the hair products. This process leaves the inner shaft of the hair dry and brittle. Inversely, by having a lower salt concentration in the conditioner than the scalp and hair cortex, the claimed invention can induce osmosis into the hair shaft and scalp, resulting in more hydrated, healthier, and stronger hair.

Branched chain amino acids (BCAA) are 3 of the 20 naturally occurring amino acids. The BCAA content determines the oily nature of all proteins. When added to a composition for hair application, BCAAs can embed into the hair locking in moisture for longer periods of time.

Follicle stimulating amino acids (FSAA) include L-lysine and N-acetyl cysteine. N-acetyl cysteine has been shown to prevent hair loss resulting from chemical damage. It is also used as an edible dietary supplement. Acetylation increases the membrane permeability of cysteine which is necessary to increase its scalp penetration. L-Lysine has been found to also be a follicle stimulating amino acid (Rushton, D. H. (2002) Clinical and Experimental Dermatology). It has also been found that high levels of L-Lysine can make cells impervious to viruses, and that long term use of L-Lysine is great for health.

L-arginine can be used alternatively as a FSAA (Fossel, E. T., 1999, U.S. Pat. No. 5,895,658 A). However, L-arginine is a vasodilator. It is known to swell hair fibers but can leave hair fibers shriveled and weak after discontinued use. Therefore, it is not preferred to use a molar ratio of L-arginine:L-lysine greater than 1:1. This is because if arginine levels exceed lysine levels it can make the scalp more susceptible to viral infection. Also, L-arginine is more basic than L-lysine and therefore more harsh on organic tissue.

The composition of the present invention is basic enough to detangle/straighten hair effectively but mild enough to prevent scalp and cuticle damage. Therefore the pH may be between 9-12, more preferably between 9 and 11, and even more preferably around 10.

In contrast to the composition described in Lissner et al., (2012) WO 2012/163594 A2 that uses unnatural synthetic alkanolamines (not all-natural) for waving/straightening hair and cysteine, the composition of the present invention disclosed herein uses only natural follicle stimulating amino acids for hair growth and waving, and N-acetyl cysteine. Cysteine has comparatively poor water solubility and is thought to have less membrane permeability. For these reasons most dietary supplementation of cysteine is done with N-Acetyl cysteine.

In certain embodiments, the composition comprises oil, which refers to any neutral, nonpolar viscous liquid that is hydrophobic and lipophilic, such as for example, jojoba oil. Certain embodiments teach that the composition includes an emulsifying wax, which is a wax-based ingredient that is a mixture of two or more liquids that are usually unmixable or immiscible. Emulsifying wax is typically generated when wax is treated with a detergent such as for example, sodium dodecyl sulfate, to generate a smooth emulsion. Wax includes but is not limited to vegetable-derived wax (such as Milliard NON-GMO Emulsifying Wax), beeswax, plant based wax and petroleum based wax. The other ingredients in the emulsifying wax may include but are not limited to Polysorbate 60, PEG-150 Stearate, Steareth-20 and Cetearyl Alcohol.

Inactive ingredients present in the composition may include but are not limited to coconut oil, canola oil, olive oil, corn oil, soybean oil, safflower oil, and avocado oil.

### EXAMPLES

Formulation 1

The composition for hair application containing only amino acids may have the following composition: Valine (1 g), Isoleucine (1 g), Leucine (1 g), N-acetyl cysteine (30 g) and l-lysine (50 g) and deionized or distilled water (100 g). The resulting solution is heated and stirred until dissolved.

Formulation 2

The composition for hair application may be mixed or made in situ with oil and emulsifying wax to make a cream for application. An example of the in situ preparation of cream is as follows: Valine (2 g), Isoleucine (2 g), Leucine (2 g), N-acetyl cysteine (60 g, 0.18 mole) and l-Lysine (100 g, 0.28 mole) and water (240 g). To this mixture jojoba oil (100 ml) and emulsifying wax (30 g) were added. The resulting suspension was heated and stirred until dissolved to yield a yellowish white emulsion. The emulsion was allowed to cool at room temperature while stirring to prevent separation. The amount of oil and emulsifying wax can be adjusted to change the thickness of the application cream.

Formulation 3

The composition of the claimed invention may contain bicarbonates and be prepared as follows: N-acetyl cysteine (30 g, 0.18 mole) and sodium bicarbonate (30 g, 0.35 mole) were added to 100 g of water. The resulting solution was heated and stirred until dissolved.

Formulation 4

The composition of the claimed invention may contain carbonates and be prepared as follows: N-acetyl cysteine (30 g, 0.18 mole) and potassium carbonate (30 g, 0.21 mole) were added to 100 g of water. The resulting solution was heated and stirred until dissolved.

Formulation 5

In some embodiments, the composition of the claimed invention is an aqueous solution containing cysteine and/or any water soluble derivative (10-25% w/w) and any mixture of glycine, lysine, arginine, glutamine, glutamate (with an esterified side chain), aspartate (with an esterified side chain), aspargine, serine, proline, tryrosine, hisitidine, threonine, or methionine at (15-40% w/w) and/or any water soluble derivative of these amino acids. The pH can be buffered by alkali and/or alkaline earth phosphates, bicarbonate, and/or carbonates.

Formulation 6

In some embodiments, the composition of the claimed invention is an aqueous solution containing N-acetyl cysteine and/or any of its water soluble derivatives (1-5 M) and sodium, potassium, calcium, and/or lithium bicarbonate/carbonate (0.5-8 M). An example of the claimed invention with only amino acids is as follows: N-acetyl cysteine (30 g, 0.18 mole) and l-arginine (50 g, 0.28 mole) were added to 100 g of water. The resulting solution was heated and stirred until dissolved.

In one embodiment, the composition is used as an anti-breakage/shedding conditioner. In order to apply the composition, the hair is washed with a standard conditioner and/or shampoo. The composition is applied to wet hair and hair is allowed to dry. The hair is noticeably stronger and softer after the application. In another embodiment, the composition is used as hair detangler using the following procedure. Hair is washed thoroughly, the composition is applied and left to process for 30-45 minutes. Then, hair is gently combed or brushed through. Finally, the composition is washed out of the hair. In some embodiments, the composition is used as a curl loosening agent in the following manner. The hair is washed thoroughly, the composition is applied to hair and a plastic cap is placed on hair. The processed hair is dried using a hair drier for 30-60 minutes at medium heat. Intermittently, the hair is checked and combed through every 10 minutes. The hair is then removed from the hair drier and gently detangled. The composition is then washed out of the hair. In still further embodiments, the composition is used as a heat free hair straightening agent in the following manner. The hair is washed thoroughly, the composition is applied to hair and a plastic cap is placed on hair. The composition is left to process for 9 hours or overnight. The hair is gently combed and the composition is washed out of the hair.

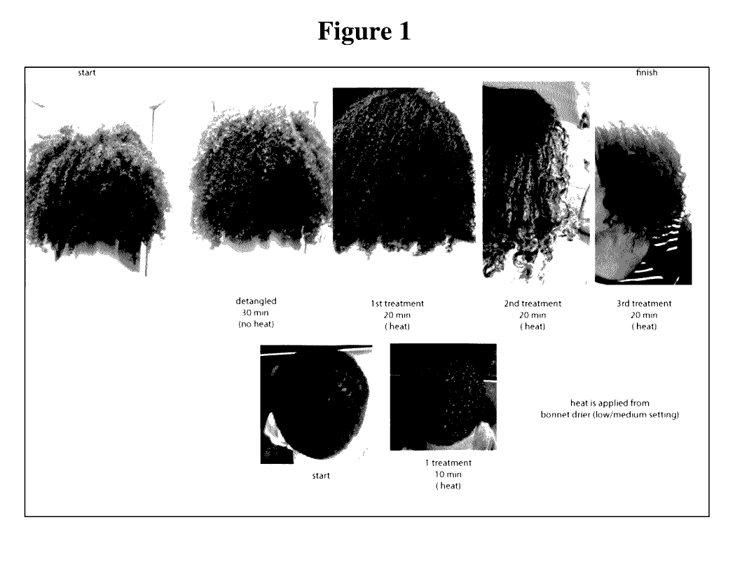

Representative examples of hair detangling effects and hair straightening effects of the claimed invention are shown in FIG. 1.

The application of the composition has been described in the form of certain embodiments and examples. However, the embodiments described herein illustrate the principles of the embodiments of the application and therefore are not limited to precisely what is described. It is understood that one of ordinary skill in the art can modify the disclosed embodiments to attain alternative or variant embodiments with alternative or variant usages, all of which are covered by the present application and can be within the scope of the application.

Further, the different elements and steps discussed above, and equivalents thereof may be used in any combination by one of ordinary skill in the art. It is also understood that some of the various elements and steps described herein may be specifically included or excluded in diverse embodiments. All methods described herein may be carried out in any order unless other indicated or contraindicated by context.

Many procedures described provide several ways to carry out the application. It is understood by one of ordinary skill in the art that not all uses or benefits may be attained from any one particular embodiment. The methods may be carried out in a way that optimizes one or one group of benefits without achieving other benefits described herein.

The terms “a”, “an” and “the”, and similar references used in the context of describing a composition, formulation or method or a particular embodiment may cover both the singular and the plural. The recitation of value ranges, such as for example, pH ranges, is intended to refer to each separate value within the range individually. Each such value is incorporated into the specification unless otherwise indicated.

The use of any and all examples and exemplary language used in the context of various embodiments is intended to only better illustrate the applicability of the composition, and does not pose a limitation on the scope of the application as claimed. Preferred embodiments including the best mode known to the inventor are described herein.

All patents, patent applications, publications of patent applications and other material such as articles, books, publication and/or the like, referenced in the application are hereby incorporated herein by this reference in their entirety. This excludes any prosecution file history that is inconsistent with or in conflict with the present application or that may have a limiting effect as to the broadest scope of the claims now or later associated with the present application. For example, if there is any inconsistency or conflict between the any of the incorporated material and the present application, the present application shall prevail.

What is claimed is:

1. A composition for hair application comprising water, at least one branched-chain amino acid, and at least one follicle-stimulating amino acid, wherein a pH of the composition is within the range of 9-12.

2. The composition of claim 1, wherein the at least one branched-chain amino acid is selected from the group consisting of leucine, isoleucine and valine

3. The composition of claim 1, wherein the at least one follicle-stimulating amino acid is selected from a group consisting of L-lysine and N-acetyl cysteine.

4. The composition of claim 1, wherein the at least one branched-chain amino acid is a D-branched-chain amino acid, an L-branched-chain amino acid, or a combination thereof.

5. The composition of claim 1, wherein the at least one follicle-stimulating amino acid amino acid is a D-follicle-stimulating amino acid, an L-follicle-stimulating amino acid, or a combination thereof.

6. The composition of claim 1, wherein the composition is substantially free of inorganic salts.

7. The composition of claim 1, wherein the water is distilled or deionized.

8. The composition of claim 1, further comprising oil.

9. The composition of claim 8, wherein the oil is jojoba oil, coconut oil, canola oil, olive oil, corn oil, soybean oil, safflower oil, avocado oil or a combination thereof.

10. The composition of claim 1, further comprising emulsifying wax.

11. The composition of claim 10, wherein the emulsifying wax is vegetable-derived wax, beeswax or a combination thereof.

12. The composition of claim 1, wherein the at least one branched-amino acid is used in an amount of from 5 to 15 parts by weight based on 100 parts by weight of water.

13. The composition of claim 1, wherein L-lysine is used in an amount of from 1 to 50 parts by weight based on 100 parts by weight of water.

14. The composition of claim 1, wherein the N-acetyl cysteine is used in an amount of from 25 to 30 parts by weight based on 100 parts by weight of water.

15. A method for preventing, reducing or alleviating hair breakage, hair shedding or a combination thereof, the method comprising applying on the hair an effective amount of the composition of claim 1.

16. The method of claim 15, wherein the hair is mammalian hair.

17. The method of claim 16, wherein the mammalian hair is human hair.

18. The method of claim 15, wherein the hair is injured, damaged, tangled, dry, brittle, healthy or a combination thereof.

19. The method of claim 15, wherein the hair is curly, wavy, straight, or a combination thereof.

20. A method for hair strengthening, hair softening, hair detangling, hair curl loosening, hair straightening, hair growth, hair hydration or a combination thereof, the method comprising applying on the hair an effective amount of the composition of claim 1.

21. The method of claim 20, wherein the hair is mammalian hair.

22. The method of claim 21, wherein the mammalian hair is human hair.

23. The method of claim 20, wherein the hair is injured, damaged, tangled, dry, brittle, healthy or a combination thereof.

24. The method of claim 20, wherein the hair is curly, wavy, straight, or a combination thereof.
